(19) United States
(12) Patent Application Publication
(10) Pub. No.: US 2004/0101501 A1
(43) Pub. Date: May 27, 2004

(54) Water-in-oil high internal phase hair relaxer composition

(75) Inventors: Liang Bin Chen, Hoffman Estates, IL (US); Shimei Fan, Inverness, IL (US); Zhuning Ma, Schaumburg, IL (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/302,660

(22) Filed: Nov. 22, 2002

(51) Int. Cl. A61K 7/06; A61K 7/11; A61K 7/09

(52) U.S. Cl. 424/70.12

(57) ABSTRACT

An water-in-oil hair relaxing composition comprising:

a) about 40% to about 95% of an aqueous phase comprising (i) water and (ii) a hair relaxing agent;

b) about 5.0 to about 30% of an oil phase, comprising a member selected from the group consisting of a volatile silicone compound, a nonvolatile silicone compound, a volatile hydrocarbon compound, a nonvolatile hydrocarbon compound, and mixtures thereof;

c) about 0.3% to about 20% of an emulsifier selected from the group consisting of a silicone-free emulsifier; a blend of silicone-free emulsifiers, having an HLB value of about 10 or less; a silicone-based emulsifier; a polymeric emulsifier; and mixtures thereof; is described.

The invention also relates to methods for relaxing the hair which comprise contacting the hair with compositions of the invention.

### BACKGROUND OF THE INVENTION

Currently marketed hair relaxers cause scalp irritation because of the direct contact between the alkaline active material and the scalp. The alkaline materials which are often used to break the disulfide bonds in hair and thereby relax the hair can be NaOH and LiOH.

Because currently marketed hair relaxers contain water as the continuous phase and have dissolved therein alkaline materials such as NaOH and LiOH, which break the disulfide bonds in hair and thereby relax the hair, the direct contact between these alkaline materials and the scalp often leads to scalp irritation and irritation of other parts of the skin.

It would be desirable to provide a hair relaxing composition wherein the active alkaline materials do not directly contact the scalp or skin. Scalp and skin irritation would thereby be avoided.

The following patents and patent applications are related to the field of the present invention.

U.S. Pat. No. 5,304,370 discloses a hair relaxer composition comprising a mixture of:

a) a base composition containing a water phase comprised of sodium hydroxide in water, and an oil phase, wherein the water phase is dispersed in the oil phase in the form of water in oil emulsion micelles,

b) an additive composition comprised of a water in oil emulsion whereby the mixture of (a) and (b) is an emulsion within an emulsion.

WO 8909048 discloses a highly alkaline, no-base hair relaxer cream composition which is phase-stable on aging. A composition of this invention is a highly alkaline cosmetic cream base that may be used directly as a no-base hair relaxer or as a no-lye cream that may be converted for use as a no-base, no-lye hair relaxer by admixture with an aqueous activator solution. A method for preparing such a phase-stable no-base hair relaxer cream composition is also disclosed.

U.S. Pat. No. 5,589,177 discloses rinse-off, water-in-oil-in-water multiple emulsion compositions. The multiple emulsion compositions comprise an external aqueous phase optionally incorporating an emulsifier and/or a second topically-active compound. The internal phase comprises a primary water-in-oil emulsion, wherein the primary emulsion comprises a first topically-active compound, a surfactant phase, an oil phase, and water.

WO 9424987 relates to hair treatment formulations such as hair relaxers, permanent waving agents and depilatories. These formulations are produced utilizing a microemulsion. While it would be expected that such microemulsions would be more irritating than their macroemulsion counterparts, the opposite has been found to be true.

U.S. Pat. No. 6,235,275 discloses opaque hair conditioner compositions that have a lamellar dispersion in the internal aqueous phase and which are water in oil emulsions that comprise:

a) about 40 to 95% aqueous phase comprising (i) water and (ii) a cationic surfactant, capable of forming lamellar dispersion, present at about 0.1 to about 10% of the aqueous phase;

b) about 0.5 to about 30% oil, comprising a volatile silicone compound, a nonvolatile silicone compound, a volatile hydrocarbon compound, a nonvolatile hydrocarbon compound, or a mixture thereof;

c) about 0.1 to about 20% silicone surfactant; and

d) optionally di-long chain alkyl amines having C 10 to C 22 carbon chains, long chain fatty alcohols having C 10 to C 22 carbon chains, or ethoxylated fatty alcohols.

### SUMMARY OF THE INVENTION

The invention relates to compositions that have a hair relaxing agent in the internal aqueous phase.

A purpose of the invention is to provide a hair relaxer with improved performance and which causes less irritation to the skin or scalp.

More specifically, the present invention relates to compositions which are water-in-oil emulsions which comprise

a) about 40% to about 95% of an aqueous phase comprising (i) water and (ii) a hair relaxing agent;

b) about 5.0 to about 30% of an oil phase, comprising a member selected from the group consisting of a volatile silicone compound, a nonvolatile silicone compound, a volatile hydrocarbon compound, a nonvolatile hydrocarbon compound, and mixtures thereof;

c) about 0.3% to about 20% of an emulsifier selected from the group consisting of a silicone-free emulsifier; a blend of silicone-free emulsifiers, having an HLB value of about 10 or less; a silicone-based emulsifier; a polymeric emulsifier; and mixtures thereof.

The invention also relates to methods for relaxing the hair which comprise contacting the hair with compositions of the invention.

### DETAILED DESCRIPTION OF THE INVENTION

Unless indicated otherwise, as used herein, % means weight % of the entire composition. In certain instances, % means weight % of a particular phase of a composition, and this is so indicated. For example, the % of surfactant is often expressed herein as the weight % of the entire amount of the aqueous phase. The word “emulsifier” and “surfactant” are used interchangeably in the present invention. The words “thickening agent” and “gelling agent” are used interchangeably in the present invention. The starting materials set forth herein are either known or can be prepared in accordance with known methods.

More specifically, the present invention relates to compositions which are water-in-oil emulsions which comprise

a) about 40% to about 95% of an aqueous phase comprising (i) water and (ii) a hair relaxing agent;

b) about 5.0 to about 30% of an oil phase, comprising a member selected from the group consisting of a volatile silicone compound, a nonvolatile silicone compound, a volatile hydrocarbon compound, a nonvolatile hydrocarbon compound, and mixtures thereof;

c) about 0.3% to about 20% of an emulsifier selected from the group consisting of a silicone-free emulsifier; a blend of silicone-free emulsifiers, having an HLB value of about 10 or less; a silicone-based emulsifier; a polymeric emulsifier; and mixtures thereof.

The aqueous phase may be present in a range of about 70 to about 90%, more preferably at about 75 to about 85% of the total composition.

Component a(ii) may be present at about 0.1 to about 4% of the total composition, more preferably at about 0.5 to about 2%.

Preferably, the present invention relates to compositions which are water-in-oil emulsions as described above which comprise:

i) about 0.5% to about 2% of the hair relaxing agent;

ii) 0 to about 15% silicone oil or 0 to about 15% hydrocarbon oil, or mixtures thereof, with the proviso that the silicone oil and the hydrocarbon oil cannot both be 0%; and

iii) about 0.4% to about 7% silicone emulsifier or silicone-free emulsifier or mixtures thereof.

The oil phase of these compositions comprises ingredients b and c. The aqueous phase comprises ingredients a.

The compositions of the invention have both high viscosity and opacity through the use of a high internal phase water-in-oil emulsion with the hair relaxing agent inside the internal water phase. These compositions deliver hair relaxing agent onto the hair.

Another aspect of the invention is to prevent direct contact of hair relaxing agent on the scalp or skin. Unlike conventional hair relaxer, which is water continuous, the present invention is oil continuous. The relaxing agent is enclosed inside the internal phase water droplets. If accidental contact occur between hair relaxing agent and scalp, the active alkaline materials do not directly contact the scalp. Scalp irritation would thereby be avoided.

When hair relaxer composition of the present invention is applied onto hair, upon the application of shear, the emulsion droplets will break and release the active alkaline materials, NaOH or LiOH, to break the disulfide bonds and thereby relax the hair.

What now follows is a description of the ingredients which may be employed in the compositions of the invention.

### HAIR RELAXING AGENTS

Compositions of the present invention may include a hair relaxing agent. A hair relaxing may be any hydroxide material which has sufficient solubility in water to give a pH in the range between 12 to 14 at ambient temperature. For example: NaOH, LiOH and Ca(OH)₂. The pH range of aqueous droplets which are hair relaxers in compositions of the invention is typically about 12 to about 14.

### OIL PHASE

The silicone oil phase of the compositions of the invention comprises a volatile silicone oil, a nonvolatile silicone oil phase or a mixture thereof preferably, the volatile silicone compound. Exemplary volatile silicone compounds include, but are not limited to, volatile, low molecular weight polydimethylsiloxane compounds. They can be either a linear or a cyclic polydimethylsiloxane compound having a viscosity of about 0.5 to 10 cst (centistokes). The preferred volatile polydimethylsiloxanes have a viscosity in the range of about 0.5 to about 6 cst.

The cyclic, volatile, low molecular weight polydimethylsiloxanes, designated in the CTFA Dictionary as cyclomethicones, are preferred siloxanes which may be used in compositions of the present invention.

These volatile compounds have an average of about 3 to about 6-[O-Si (CH₃)₂]-repeating group units per molecule (hexamethylcyclotrisiloxane, octamethylcyclotetrasiloxane, decamethylcyclopentasiloxane, dodecamethylcyclohexasiloxane, and mixtures thereof); and boil at atmosphere pressure at about 150° C. to about 250° C. The polydimethyl cyclosiloxanes having an average of about 4 to about 5 repeating units per molecule are especially preferred. Suitable cyclomethicones are available commercially under the trade names DOW CORNING 245 Fluid, DOW CORNING 344 Fluid and DOW CORNING 345 Fluid from DOW CORNING Corporation, Midland, Mich., and SILICONE SF-1173 and SILICONE SF-1202 from General Electric, Waterford, N.Y.

An example of a linear, low molecular weight, volatile polydimethylsiloxane compound is designated in the CTFA Dictionary as decamethyltetrasiloxane, available commercially under the trade names DOW CORNING 200 Fluid having a viscosity of 1.5 cst and a boiling point of 195° C. Other linear polydimethylsiloxanes includes octamethyltrisiloxane, and decamethylpentasiloxane which may also be useful in the compositions of the present invention.

A volatile hydrocarbon oil phase comprises about 6 to 20 carbon atoms. A preferred volatile hydrocarbon compound is an aliphatic hydrocarbon having about 8 to about 16 carbon atoms, and having a boiling point of about 100 to about 250° C. Exemplary volatile hydrocarbon compounds may include, isododecane and isohexadecane, that is, PERMETHYL 99A, PERMETHYL 101A, available from Presperse, Inc., South Plainfield, N.J. Other examples of hydrocarbon compounds which may be useful in compositions of the invention are depicted in general formula below, wherein n ranges from 2 to 3.

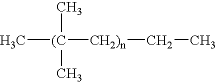

Another exemplary volatile hydrocarbon compound is ISOPAR M (a C₁₂-C₁₄ isoparaffin available from EXXON Chemical Co., Baytown, Tex.).

Exemplary nonvolatile silicone compounds include a polydimethylsiloxane, polyalkyl siloxane, a polyaryl siloxane or a polyalkylaryl siloxane. Nonvolatile silicones are nonfunctional siloxanes or siloxane mixtures having a viscosity of about 10 to about 10,000 cst, and most preferred viscosity about 10 to about 500 cst at about 25° C. A nonvolatile silicone compound having a boiling point at atmospheric pressure of greater than about 250° C. Phenyltrimethicone may also be useful as a nonvolatile silicone compound. Examples include DC 556 fluid, which is available from Dow Corning.

The nonvolatile oil phase may also comprise a nonvolatile hydrocarbon compound, such as petrolatum, mineral oil, benzoate or isoeicosane. Other exemplary compounds includes water insoluble emollient, such as, for example, an ester having at least about 10 carbon atoms, and preferably about 10 to about 32 carbon atoms. Suitable esters for use in compositions of the invention include but are not limited to, for example, aliphatic monohydric alcohol esters including isopropyl myristate and aliphatic di- or tri-esters of polycarboxylic acids including dioctyl adipate. Suitable nonvolatile hydrocarbon compounds also include stearyl alcohol or isostearyl alcohol.

As noted above, in the present invention, the compositions have a high internal phase water-in-oil emulsion with the active alkaline material inside the internal water phase. These compositions deliver hair relaxing agent onto the hair. The appearance, viscosity, texture and stability of the emulsion depends greatly on the type of emulsifier. For this present invention, a wide variety of emulsifier can be used. To achieve the full advantage of the present invention, about 0.1% to about 20% by weight of the emulsifiers is used. The emulsifiers can comprise a silicone-free emulsifier, or a blend of silicone-free emulsifiers, having an HLB value of about 10 or less (i.e. an HLB value of about 0.1 to about 10), a silicone-based emulsifier, a polymer emulsifier or mixtures thereof. Preferably the silicone-free emulsifier or emulsifier blend has an HLB value of about 3 to about 6.

The emulsifier therefore comprises a single silicone-free emulsifier having an HLB value of about 0.1 to about 10, or a blend of silicone-free emulsifiers having different HLB values such that the blend has an HLB values of about 1 to about 10. The emulsifier for the present invention can also comprise a blend of a silicone-base surfactant, or a polymeric emulsifier, and a silicone-free emulsifier having an HLB value of about 10 or less. The above-described emulsifier system provide a water-in-oil emulsion.

In general, the emulsifier comprises a silicone-based emulsifier when the oil phase comprises a volatile or a nonvolatile silicone compound. If the oil phase is a volatile or nonvolatile hydrocarbon compound, the emulsifier preferably comprises a silicone-free non-ionic surfactant or a polymeric surfactant. If a combination of a silicone compound and hydrocarbon compound is used as the oil phase, the emulsifier system preferably comprises a combination of (1) a silicone-based emulsifier and (2) a silicone-free emulsifier, a polymeric surfactant or a mixture thereof.

### Silicone-Free Emulsifier

A silicone-free nonionic surfactant having an HLB value of about 0.1 to about 10 can be used alone as the emulsifier of the present invention. The emulsifier system also can comprise a blend of silicone-free surfactants each having an HLB value of less than 10. In addition, the silicone-free emulsifier having an HLB value of about 0.1 to about 10 also can be used as the first surfactant or a surfactant blend having an HLB value of about I to about 10, then a silicone-free surfactant having an HLB of greater than 10 is the second surfactant of the surfactant blend having an HLB of about 1 to about 10.

Typically, silicone-free nonionic surfactants having an HLB value of about 10 or less have a hydrophobic moiety, such as a long chain (C₈-C₂₂) alkyl group or an alkylated aryl group, and a hydrophilic chain comprising a small number (i.e. one to about six) of ethoxy moieties or a combination of ethoxy and propoxy moieties. The silicone-free nonionic surfactants having an HLB of greater than about 10 typically have the same type of hydrophobic moiety as the low HLB surfactants, but include more ethoxy and/or propoxy moieties.

The HLB value of a particlular silicone-free emulsifier can be found in McCutcheon's Emulsifiers and Detergents, volume 1, North American Editions, MC Publishing Co., Glen Rock, N.J. (2001) (hereinafter McCutcheon's). Alternatively, the HLB value of a particular surfactant can be estimated by dividing the weight percent of oxyethylene in the surfactant by five (for surfactants including only ethoxy moieties). In addition, the HLB value of a surfactant blend can be estimated by the following formula:

HLB=(wt. % A)(HLB_{A})+(wt. % B)(HLB_{B})

Wherein wt. % A and wt. % B are the weight percent of surfactants A and B in the silicone-free surfactant blend, and HLB_{A} and HLB_{B} are the HLB values for surfactants A and B, respectively.

Exemplary classes of silicone-free nonionic emulsifiers include, but are not limited to, polyoxyethylene ethers of fatty (C₆-C₂₂) alcohols, polyoxyethylene/polyoxypropylene ethers of fatty (C₆-C₂₂) alcohols, ethoxylated alkylphenols, polyethylene glycol ethers of methyl glucose, polyethylene glycol ethers of sorbitol, and mixture thereof.

Exemplary silicone-free nonionic emulsifiers are the ethoxylated alcohols having an HLB value of about 0.1 to about 10. An especially preferred ethoxylated alcohol is laureth-1, that is, lauryl alcohol ethoxylated with an average of one mole of ethylene oxide. Other suitable ethoxylated alcohols include laureth-2, laureth-3, and laureth-4. Numerous other nonionic emulsifiers having an HLB of about 0.1 to about 10 are listed in McCutcheon's at pages 251-257, incorporated herein by reference. Other exemplary silicone-free nonionic surfactants having HLB value of about 0.1 to about 10 includes, but are not limited to, the ethoxylated nonylphenols, ethoxylated octylphenonis, ethoxylated dodecylphenols, ethoxylated fatty (C₆-C₂₂) alcohols having four or fewer ethylene oxide moieties, oleth-2, steareth-3, steareth-2, ceteth-2, oleth-3, and mixtures thereof.

The emulsifier system for the present invention also can comprise a silicone-free surfactant blend having an HLB value of about 1 to about 10. The blend is a mixture of a sufficient amount of a surfactant having a low HLB value, i.e., about 0.1 to about 10, and a sufficient amount of a surfactant having a higher HLB value, i.e., about 1 to greater than about 10, such that the surfactant blend has an HLB value of about 1 to about 10, sucha that the surfactant bloend has an HLB value of about 1 to about 10. Exemplary, but nonlimiting, nonionic surfactants having a high HLB value are listed in McCutcheon's at pages 257-267, incorporated herein by reference.

Exemplary silicone-free nonionic surfactants having an HLB value greater than about 10 are oleth-1 0, octylphenol or nonylphenol ethoxylated with six or more moles of ethylene oxide, steareth-10, tedieceth-6, methyl gluceth-10, dodoxynol-12, ceteth-12, C₁₁₋₁₅ pareth-20, and mixtures thereof. The identity of the high HLB surfactant is not limited as long as the resulting surfactant phase provides a stable water-in-oil emulsion.

Suitable silicone-free nonionic surfactants such as polyethoxylated alcohols, ethoxylated phenols, ethoxylated fatty acid and esters are suitable emulsifiers for the present invention. Esters such as polyglycerol-3 diisostearate, polyglycerol-2 diisostearate and polyglycerol-2 triisostearate can also be used as the emulsifiers for the present invention.

### Silicone Emulsifier

Exemplary of the silicone surfactants or emulsifiers that may be used in compositions of the invention is a dimethicone, which is a dimethylsiloxane polymer having polyoxyethylene and/or polyoxypropylene side chains, such as DOW CORNING 3225C and 5225C FORMULATION AID, available from Dow Corning Co., Midland, Mich., ABIL EM 97, available from Goldschmidt Chemical Corporation, Hopewell, Va. and SILWET™ series, available from OSI Specialties, Inc., Danbury, Conn. The dimethicone copolyol has about 15 or fewer ethylene oxide and/or propylene oxide monomer units, in total, in the side chains. Dimethicone copolyols conventionally are used in conjunction with silicones because the oil-soluble, silicon-based surfactants are extremely soluble in a volatile or a nonvolatile silicone compound, and are extremely insoluble in water.

These dimethicone copolyols which may be employed in compositions of the invention, can be dimethicone copolyols with HLB values of less than 10, more preferably about 2 to about 8. These dimethicone copolyols may have a molecular weight of about 600 to about 20,000. These dimethicone copolyols may be from the SILWET series such as DC 5225C and DC 3225C. Also alkyl dimethicones such as cetyl dimethicone may be used in compositions of the invention.

Dimethicone copolyols which may be used in the compositions of the invention can have the following formula:

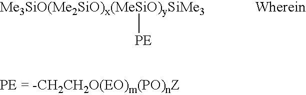

wherein Me is methyl, EO is ethyleneoxy, PO is 1,2-propyleneoxy, x and y are 1 or greater, m and n are 0 or greater, provided that the molecular weight of the PE moiety must be greater than 1,000 and Z is hydrogen or lower alkyl (like a C₁-C₅ alkyl).

Another exemplary, but non-limiting, oil-soluble, silicon-based surfactant is an alkyl dimethicone copolyol, such as cetyl dimethicone copolyol available commercially as ABIL EM 90 from Goldschmidt Chemical Corporation, Hopewell, Va. The alkyl dimethicone copolyols have the structure:

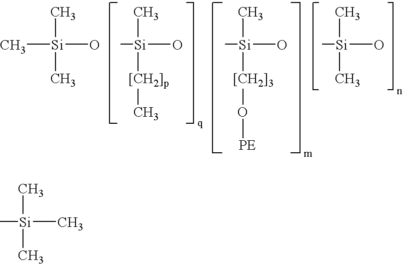

wherein p is numeral from 7 through 17;

q is a numeral from 1 through 100;

m is a numeral from 1 through 40;

n is a numeral from 0 through 200; and

PE is (C₂H₄O)ₐ(C₃H₆O)_{b}-H having a molecular weight of about 250 to about 2000, wherein a and b are selected such that the weight ration of C₂H₄O/C₃H₆O is from about 100/0 to about 20/80.

### Polymeric Surfactants

Accordingly, exemplary polymeric surfactants include, but-are not limited to, polyoxyethylene-polyoxypropylene block copolymers, and similar polyoxyalkylene block copolymers. The block copolymers typically have less than about 20% by weight of ethylene oxide. Specific nonlimiting polymeric surfactants include Poloxamer 101, Poloxamer 105, PPG-2-Buteth-3, PPG-3-Buteth-5, PPG-5-Buteth7, PPG-7-Buteth-10, PPG-9-Buteth-12, PPG-12-Buteth-16, PPG-15-Buteth-20, PPG-20-Buteth-30, PPG-24-Buteth-27, PPG-28-Buteth-35, and PEG-15 Butanediol. Other useful polymer surfactants are poloxamines, i.e., polyoxyethylene-polyoxypropylene block copolymers of ethylene diamine, having less than about 40% by weight ethylene oxide.

The invention may also comprise a thickening or gelling agent for the oil phase. Illustrated below are different types of thickening or gelling agent.

### Wax

The compositions of the present invention may contain one or more wax-like materials having a low melting point, that is, having a melting point of from about 25° C. to about 75° C. These low melting point waxes are present at from about 0.01% to about 40% of the oil phase. Such materials are well known in the art and include fatty acids, fatty alcohols, fatty acid esters, and fatty acid amides, having fatty chains of from about 8 to about 30, preferably from about 12 to about 18, carbon atoms. Preferred low melting point waxes include cetyl alcohol, palmitic acid, myristyl alcohol, stearyl alcohol, paraffin, and mixtures thereof. Stearyl alcohol, cetyl alcohol, and mixtures thereof are particularly preferred.

The low melting point waxes described above can be used with other water-insoluble waxes which are described more fully hereinafter to provide emolliency and also to control the rate of product depositing on the skin. One skilled in the art would be able to adjust the cosmetic aesthetics and physical structure by combining various suitable water-insoluble waxes with the water-insoluble liquid emollients.

The compositions of the present invention may contain one or more materials having wax-like characteristics and a melting point of from about 65° C. to about 130° C. When used, these high melting point waxes are present at from about 0.01% to about 10% of the oil phase composition. Such waxes include beeswax, carnauba, baysberry, candelilla, montan, ozokerite, ceresin, hydrogenated castor oil (castor wax), synthetic waxes such as Fisher-Tropsch waxes, microcrystalline waxes, and mixtures thereof. Castor wax is a preferred high melting point wax for use herein. High melting point waxes useful in compositions of the present invention are also disclosed in U.S. Pat. No. 4,049,792, Elsnau, issued Sep. 20, 1977, incorporated herein by reference.

Syncrowaxes such as glyceryl tribehenate, C₁₈-C₃₆ acid triglycerides and, C₁₈-C₃₆ acid glycol ester are also suitable as gelling agent for the oil phase.

### Polymer Gellant

Polyethylene (particularly 0.01-30% of the oil phase composition) comprising one or more members selected from the group consisting of homopolymers and copolymers of polyethylene may also be used as gelling agents in the compositions of the present invention. It has a polymer backbone of CH₃CH₂—(CH₂—CH₂)ₙ—CH₂—CH₃ (which can also be represented as CH₃CH₂—(CH₂—CH₂)ₙ—H), where n is an average number and is selected to be in the range of about 10 to about 106. Polyethylene is at least 90% linear and has a molecular weight in the range of about 300 to about 3000. Polyethylene has a melting point in the range of about 50 to about 129 degrees C.

Silicone-based polyamide (U.S. Pat. No. 6,353,076 which is herein incorporated by reference) and block copolymer of hydrocarbon-polyether-polyamide-polyether-hydrocarbon types (U.S. Pat. No. 6,399,713 which is herein incorporated by reference) are also suitable polymer gelling agent for the oil phase.

### Particulates

Another gelling agent which may be used in compositions of the present invention is a finely divided silica material (from about 0.01% to about 2% of the oil phase composition) which is comprised of micron to sub-micron sized silica particulates with high surface area (preferably greater than about 100 square meters per gram of material). Colloidal silica materials useful herein include Syloid silicas (manufactured by Davison Chemical Division of W. R. Grace), Cab-O-Sil (manufactured by Cabot Corporation), and Aerosil (manufactured by Degussa A. G.). Cab-O-Sil, having a surface area of from about 200 to about 400 square meters per gram, is a particularly preferred commercially-available colloidal silica useful in compositions of the present invention.

### Small Molecular Gelling Agent

The oil phase of the compositions of the present invention may also comprise small molecular gelling agents such as simple Bolaform amides derived from amino acids, cholestanyl substituted quaternary ammonium salts.

Topically-active ingredients may also be incorporated into the compositions of the present invention. These include glycerin, propylene glycol, sorbitol, proline, hyaluronate of dimethylsilanol, allantoin, aloe vera, pyrrolidone carboxylic acid, provitamine B₅, hydrogenated wheat protein, hydrolyzed keratin protein or hydrolyzing collagen.

Compositions of the invention which appear in Examples 1 through 5 below may be prepared by methods which are known to those skilled in the art. Ingredients used in the preparation of compositions of the invention are either known or may be prepared by known methods.

Compositions of the invention which appear in examples 1 through 5 below were prepared as follows:

1) Prepare the aqueous phase by mixing water and sodium hydroxide.

2) Premix the oil phase with the silicone surfactants.

3) Emulsify 1 and 2.

Occasionally, other secondary components such as, petrolatum or other long chain fatty alcohols are be incorporated into the oil phase with heating.

### High Relaxer Compositions with High Internal Phase Water-in-Oil Emulsions

Examples 1, 2 and 3 shown below were made. Make a 80 weight percent of internal aqueous phase with 1, 2 and 3 weight percent active of sodium hydroxide in the prototypes

| | No. 1 | No. 2 | No. 3 |
|---|---|---|---|
| | Wt. % | Wt. % | Wt. % |
| Water Phase | | | |
| Deionized Water | 78 | 76 | 74 |
| NaOH (50% active) | 2 | 4 | 6 |
| Oil Phase | | | |
| Cyclomethicone (D5) | 10 | 10 | 10 |
| DC 5225C | 10 | 10 | 10 |

Example 1 is a stable emulsion over a period of more than 2 weeks. Example 2 shows slight syneresis of silicone oil and Example 3 shows phase separation in less than 2 weeks.

### EXAMPLES 4 and 5

| | No. 4 | No. 5 |
|---|---|---|
| | Wt. % | Wt. % |
| Water Phase | | |
| Deionized Water | 76 | 76 |
| NaOH (50% active) | 4 | 4 |
| Oil Phase | | |
| Cyclomethicone (D5) | 11 | 2 |
| Abil EM 90 | 6 | 12 |
| DC 5225C | 3 | 6 |

Both examples 4 and 5 are stable emulsions.

The low skin and low scalp irritancy caused by examples 1 through 5 of the invention may be demonstrated through the use of conductivity measurements. Since most of the hair relaxers in the market are water continuous, their conductivity will be high. This high conductivity is due to the hair relaxing agent present in the external aqueous phase. And if accidentally this hair relaxing agent contacts the scalp and skin, irritation could result. On the other hand, compositions of the present invention are water-in-oil emulsions, which result in very low conductivity. The low conductivity indicates that the hair relaxing agent remains in the internal water phase. Therefore, when compositions of the present invention accidentally contact the scalp or skin, irritation will be minimized or avoided because the external phase in these compositions is the oil phase. The conductivity can be measured with a conductivity meter (CDM 80 from Radiometer America). The conductivity cell (CDC104 from Radiometer) has a cell constant of 0.96. The measurement is made at 25° C. For a typical water continuous hair relaxer, the conductivity can range from 1,000 to 50,000 mS/cm. However for the compositions of the present invention, the conductivity is in the range of 0.01 to 10 mS/cm. This low conductivity value indicates that the relaxing agent remains in the internal aqueous phase, and therefore that the compositions of the invention are less irritating to the skin and scalp when these compositions accidentally contact the skin or scalp.

### Exemplary Formulation

| | No. 6 |
|---|---|
| | Wt. % |
| Deionized Water | To 100 |
| NaOH (50% active) | 4 |
| Cyclomethicone (D5) | 11 |
| Abil EM 90 | 6 |
| DC 5225C | 3 |
| Glycerin | 3 |
| Hydrolyzing collagen | 0.05 |
| Fragrance | 0.3 |

| | No 7 | No 8 | No 9 | No. 10 | No. 11 |
|---|---|---|---|---|---|
| Water Phase | | | | | |
| Deionized water | 81 | 80 | 81 | 81 | 61.5 |
| NaOH 50% | 4 | 4 | 4 | 4 | 3.5 |
| Oil Phase | | | | | |
| DC 5225C 10% | 7.5 | 7.5 | | | 4.5 |
| active | | | | | |
| Cyclomethicone | 7 | 8 | | | 20 |
| Dimethicone | | | | | 5 |
| Glycerol-3 | | | 5 | 5 | |
| diisostearate | | | | | |
| Mineral oil | | | 5 | | |
| Permethyl 101 | | | | 5 | |
| Guerbet stearyl | | | 5 | | |
| alcohol | | | | | |
| Isostearyl alcohol | | | | 5 | |
| Polyethylene | | | | | 5 |
| Fragrance | 0.5 | 0.5 | | | 0.5 |

Examples 6 and 11 above are hypothetical examples of compositions of the invention. The remaining examples of compositions of the invention were made.

The invention also relates to hair relaxer kits which may contain a composition of the invention as well as instructions for its use, in a single package.

### METHODS OF THE INVENTION

Compositions of the invention are used by the consumer as follows:

1. A protective creme is first applied to the nape and skin area around the hair

2. The composition of the invention is applied using protective gloves.

3. It is applied to sections of the hair with a spatula or like instrument.

4. The spatula or like instrument is pressed against the hair strands until all or part of the hair is covered.

5. Such stroking or pressing against the hair can be repeated.

6. The hair is then rinsed with warm water at strong force until all of the composition of the invention has been removed from the hair.

What is claimed is:

1. An water-in-oil hair relaxing composition comprising:
a) about 40% to about 95% of an aqueous phase comprising (i) water and (ii) a hair relaxing agent;
b) about 5.0% to about 30% of an oil phase, comprising a member selected from the group consisting of a volatile silicone compound, a nonvolatile silicone compound, a volatile hydrocarbon compound, a nonvolatile hydrocarbon compound, and mixtures thereof;
c) about 0.3% to about 20% of an emulsifier selected from the group consisting of a silicone-free emulsifier; a blend of silicone-free emulsifiers, having an HLB value of about 10 or less; a silicone-based emulsifier; a polymeric emulsifier; and mixtures thereof.

2. A composition in accordance with claim 1 wherein the aqueous phase is present in a range of about 65% to about 90%.

3. A composition in accordance with claim 1 wherein the aqueous phase is present in a range of about 75% to about 85%.

4. A composition in accordance with claim 1 wherein the hair relaxing agent comprises hydroxide materials which have sufficient solubility in water to give a pH in the range between about 12 to about 14 at ambient temperature.

5. A composition in accordance with claim 1 wherein the hair relaxing agent is selected from the group consisting of sodium hydroxide, lithium hydroxide, calcium hydroxide and mixtures thereof.

6. A composition in accordance with claim 1 wherein component a(ii) is present at about 0.1 to about 4% of the total composition.

7. A composition in accordance with claim 6 wherein component a(ii) is present at about 0.5 to about 2% of the total composition.

8. A composition in accordance with claim 1 wherein the emulsifier is present at about 0.1% to about 15% of the total composition.

9. A composition in accordance with claim 1 wherein the emulsifier is present at about 0.3 to about 10% of the total composition.

10. A composition according to claim 1 wherein the silicone emulsifier is a dimethicone copolyol with an HLB value of less than about 10, and a molecular weight of about 600 to about 20,000.

11. A composition according to claim 1 wherein the silicone emusifier has the formula 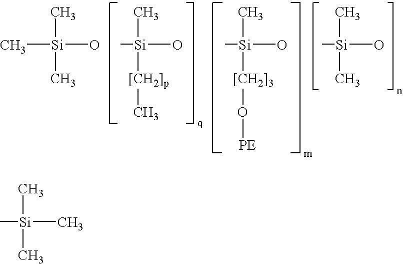
wherein p is numeral from 7 through 17;
q is a numeral from 1 through 100;
m is a numeral from 1 through 40;
n is a numeral from 0 through 200; and PE is (C₂H₄O)ₐ(C₃H₆O)_{b}-H having a molecular weight of about 250 to about 2000, wherein a and b are selected such that the weight ration of C₂H₄O/C₃H₆O is from 100/0 to about 20/80.

12. A composition according to claim 11 wherein the silicone emulsifier is cetyl dimethicone copolyol.

13. A composition according to claim 1 wherein the silicone emulsifier is a dimethicone copolyol which has the following formula: 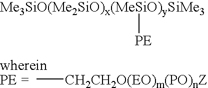
wherein Me is methyl, EO is ethyleneoxy, PO is 1,2-propyleneoxy, x and y are 1 or greater, m and n are 0 or greater, provided that the molecular weight of the PE moiety must be greater than 1,000 and Z is hydrogen or lower alkyl (like a C₁-C₅ alkyl).

14. A composition according to claim 1 wherein the oil phase comprises a member selected from the group consisting of volatile silicone oil, a nonvolatile silicone and mixtures thereof.

15. A composition according to claim 1 wherein the oil phase is a volatile silicone oil.

16. A composition according to claim 1 wherein the oil phase is a cyclomethicone.

17. A composition according to claim 1, which comprises a nonvolatile silicone compound selected from the group consisting of a polydimethylsiloxane, polyalkyl siloxane, a polyaryl siloxane, a polyalkylaryl siloxane, and mixtures thereof.

18. A composition according to claim 1 wherein the oil phase comprises a volatile hydrocarbon oil of about 6 to about 20 carbon atoms.

19. A composition according to claim 1 wherein the oil phase is a mineral oil.

20. A composition in accordance with claim 1 wherein the nonvolatile hydrocarbon compound is selected from the group consisting of comprises petrolatum, benzoate, isoeicosane, isopropyl myristate, dioctyl adipate, or mixtures thereof.

21. A composition according to claim 1 which further comprises from about 0.01% to about 40% of a wax selected from the group consisting of fatty acids, fatty alcohols, fatty acid esters, fatty acid amides, and mixtures thereof.

22. A composition according to claim 21 wherein the wax is selected from the group consisting a wax cetyl alcohol, palmitic acid, myristyl alcohol, stearyl alcohol, isostearyl alcohol, paraffin, and mixtures thereof.

23. A composition according to claim 1 which comprises a silicone-free nonionic surfactant having an HLB value of about 0.1 to about 10.

24. A composition according to claim 1 which comprises a blend of silicone-free nonionic surfactants each having an HLB value of less than 10.

25. A composition according to claim 1 which comprises a first silicone-free nonionic surfactant having an HLB value of about 0.1 to about 10 is used as the first surfactant; and which also comprises a second silicone-free surfactant having an HLB of greater than 10 an HLB of about 1 to about 10; provided that the first and second silicone-free surfactants together have an HLB of about 1 to about 10.

26. A composition according to claim 1 which comprises a silicone-free nonionic emulsifier selected from the group consisting of, polyoxyethylene ethers of fatty (C₆-C₂₂) alcohols, polyoxyethylene/polyoxypropylene ethers of fatty (C₆-C₂₂) alcohols, ethoxylated alkylphenols, polyethylene glycol ethers of methyl glucose, polyethylene glycol ethers of sorbitol, and mixtures thereof.

27. A composition according to claim 1 which comprises silicone-free nonionic surfactants selected from the group onsisting of polyethoxylated alcohols, ethoxylated phenols, ethoxylated fatty acid, ethoxylated fatty esters, and mixtures thereof.

28. A composition according to claim 1 which comprises esters sselected from the group consisting of polyglycerol-3 diisostearate, polyglycerol-2 diisostearate, polyglycerol-2 triisostearate, and mixtures thereof.

29. A composition according to claim 1 which further comprises a thickening or gelling agent for the oil phase.

30. A composition in accordance with claim 1 which further comprises a member selected from the group consisting of cetyl alcohol, palmitic acid, myristyl alcohol, stearyl alcohol, paraffin, and mixtures thereof.

31. A composition in accordance with claim 1 which further comprises a member selected from the group consisting of beeswax, carnauba, baysberry, candelilla, montan, ozokerite, ceresin, hydrogenated castor oil (castor wax), synthetic waxes such as Fisher-Tropsch waxes, microcrystalline waxes, and mixtures thereof.

32. A composition in accordance with claim 1 which further comprises a member selected from the group consisting of glyceryl tribehenate, C₁₈-C₃₆ acid triglycerides and , C₁₈-C₃₆ acid glycol, and mixtures thereof.

33. A composition in accordance with claim 1 which further comprises polyethylene.

34. A composition in accordance with claim 33 wherein polyethylene is present at about 0.01 to about 10% of the total composition.

35. A composition in accordance with claim 1 which further comprises a silicone-based polyamide, a block copolymer of hydrocarbon-polyether-polyamide-polyether-hydrocarbon polymer, or a mixture thereof.

36. A composition in accordance with claim 1 further comprises a finely divided silica material.

37. A composition in accordance with claim 1 which further comprises a member selected from the group consisting of glycerin, propylene glycol, sorbitol, proline, hyaluronate of dimethylsilanol, allantoin, aloe vera, pyrrolidone carboxylic acid, provitamine B₅, hydrogenated wheat protein, hydrolyzed keratin protein, hydroylzing collagen, and mixtures thereof.

38. A method for relaxing hair which comprises contacting the hair with a composition according to claim 1.
